(19) United States
(12) Patent Application Publication
(10) Pub. No.: US 2004/0097586 A1
(43) Pub. Date: May 20, 2004

(54) Anti-tumor compounds

(76) Inventors: Andre Trouet, Winksele (BE); Vincent Dubois, Gif sur Yvette (FR)

Correspondence Address: KNOBBE MARTENS OLSON & BEAR LLP, 2040 MAIN STREET, FOURTEENTH FLOOR, IRVINE, CA, 92614, US

(21) Appl. No.: 10/470,466

(22) PCT Filed: Jan. 30, 2002
(86) PCT No.: PCT/EP02/00951

(30) Foreign Application Priority Data
Jan. 30, 2001 (EP) EP01870017.9

(51) Int. Cl. A61K 31/205

(52) U.S. Cl. 514/554; 562/553

(57) ABSTRACT

Compounds are disclosed with the general formula A-B, which in the vicinity of tumor cells result in a positively charged moiety B and an uncharged or negatively charged moiety A. Moiety B is able to induce blood clotting by interacting with negatively charged heparin-like substances lining vascular endothelia and the positive charge is reversibly masked by the uncharged or negatively charged moiety A in order to prevent unspecific disseminated blood coagulation and toxicity. Moiety B is either a covalent assembly of positively charged chemical groups or a positively charged molecule, which in aqueous solutions forms non-covalent polycations due to its propensity to form intermolecular aggregates. Pharmaceutical compositions including the compound and a pharmaceutically acceptable adjuvant or excipient are also disclosed. The disclosed compounds are useful in medicine, in particular for the manufacture of a medicament and its use for the treatment of a subject.

### TECHNICAL FIELD

The present invention relates to novel compounds, in particular tumor-selective intravascular coagulation inducing molecules and to their pharmaceutical use. The molecules are prodrugs carrying a polycation moiety and able to induce blood clotting at the tumor sites resulting in the disruption of the tumor vascularization and consequently In the control of tumor growth.

### BACKGROUND ART

Cancer is currently the second largest killer in the developed world with more than 6 million deaths per year, a figure that is expected to double by 2002. Despite the huge efforts made for many years to improve the efficacy of treatment, relatively low cure rates are achieved in most instances. The vast majority of available therapies consist of drugs selected or designed to act on rapidly dividing cells. Besides the fact that most cancers are diagnosed at a time when the proportion of cycling (dividing “target” cells) is already very much reduced, there are two main reasons that can explain those failures. First, a number of normal tissues also contain rapidly dividing cell populations that are killed by anticancer agents. Because of the resulting severe toxicities clinicians are forced to reduce the dose levels used, as well as the frequency of treatments. Of course, this greatly impairs the efficacy of these therapies. Second, tumor cells are genetically unstable and have high mutation rates. The evolving heterogeneity of the cell population that makes a tumor explains the observation that tumors almost always develop resistance to treatment.

New treatments that are more specific for tumor cells and not subject to the development of resistance are therefore highly desirable.

Tumor-activated prodrugs that overcome toxicity as described above have been disclosed for example in Patent Cooperation Treaty International publication No WO 96/05863 and in U.S. Pat. No 5,962,216, both incorporated herein by reference.

Strategies that aim at disrupting tumor angiogenesis are currently being developed (Harris, 1997; Boehm et al., 1997; Zetter, 1998). As a matter of fact, solid tumor growth and metastasis are absolutely dependent on the formation of new blood vessels, and prevention of neoangiogenesis blocks these mechanisms and can even result in tumor shrinking and disappearance. In this case the targets are not the tumor cells themselves, but the endothelial cells responsible for tumor neoangiogenesis. Since these endothelial cells are normal, genetically-stable cells, resistance development is not anticipated.

The development of antiangiogenic drugs as anticancer agents has recently received a great impetus. Such agents impair the formation of neocapillaries, which are essential for solid tumor growth as soon as it reaches a diameter greater than 1 mm, and likely work by stopping nutrient and oxygen supply to cancer cells. This type of approach should allow to solve the major problem of innate and acquired resistance (antiangiogenic agents target normal, genetically stable endothelial cells) so frequently observed with classical cytostatic and cytotoxic anticancer agents. Antiangiogenic treatments should also be much less toxic than classical chemotherapy, but given that prolonged (perhaps life-long) treatments will very likely be required, this remains an important unanswered question and tumor-specific antiangiogenic treatments are very likely to help (Harris, 1997; Boehm et al., 1997; Molema et al., 1998).

Antitumor anthracyclines (daunorubicin and doxorubicin) have been shown for a while to form aggregates in solution as a result of the stacking of their tetracyclic moieties (Dalmark and Storm, 1981; Menozzi et al., 1984; Confalonieri et al., 1991). In a number of cases they seemed to have procoagulant activities (Wheeler and Geczy 1990, Walsh et al., 1992, Fujihira et al., 1993). Doxorubicin binds to heparin-like substances that cover the luminal surface of the endothelium of all bloodvessels, thereby inhibiting their anticoagulant properties (Cofrancesco et al., 1980; DeLucia III et al., 1993; Colombo et al., 1981, Mizuno et al., 1995). It has been shown that polycations carrying a high charge density interact with heparin-like substances causing severe toxicity in animals (Lijnen et al., 1983; Horrow, 1985; Koslow et al., 1987; Lindblad, 1989; Roelofse and van der Bijl, 1991; Tainsh et al., 1992; DeLucia III et al., 1993; Ekrami and Shen, 1995; Wakefield et al., 1992).

The coagulation-inducing properties of ETAP (Extracellularly Tumor-Activated Prodrug) compounds are not described in WO96/05863 nor in U.S. Pat. No. 5,962,216, but in WO00/33888. Said three patent applications are hereby incorporated by reference. ETAP-prodrugs can be defined as peptidic conjugates of anticancer agents (e.g. doxorubicin) stable in body fluids and normal tissues but unable to enter cells, whether normal or tumoral. Interestingly, these ETAP compounds can be cleaved extracellularly into an active form of the drug by one or more peptidases released by tumor cells allowing a more selective treatment of the tumor site. That ETAP compounds can coagulate is illustrated in WO00/33888 to be an adverse effect. Surprisingly, the combination of both principles made the basis of this new concept as described by present inventors and allow to design new prodrugs with improved characteristics.

There is a need for new cancer treatments and strategies. Treatment strategies that use anti-angiogenesis compounds appear in the prior art. There are several approaches to developing anti-angiogenic treatments and someone skilled in the art, having overcome the problem of solubility and delivery, might be expected to develop therapeutic agents that bind to or inhibit specific endothelial receiver molecules. The use of tumor-selective intravascular coagulation as described by this invention and compounds that achieve it is not obvious and represents a new strategy which does not appear in the prior art.

### DETAILED DESCRIPTION OF THE INVENTION

The aim of the present invention is to find a compound with superior characteristics as compared to previously described compounds that can be used to induce intravascular coagulation restricted to the capillaries of solid tumors and their metastases thereby preventing nutrient and oxygen supply of the cancer cells. The term ‘superior’ refers to a higher specificity towards tumors and having less toxic effects in the animal.

The present invention describes a compound of the general formula A-B, which in the vicinity of tumor cells or endothelial cells involved in tumor angiogenesis results in a positively charged moiety B and an uncharged or negatively charged moiety A, whereby said moiety B is able to induce blood clotting by interacting with negatively charged heparin-like substances lining vascular endothelia and whereby the positive charge is reversibly masked by the uncharged or negatively charged moiety A in order to prevent unspecific disseminated blood coagulation and toxicity.

That positively charged molecules can cause acute toxicity has already been demonstrated in WO00/33888 by injectng intravenously oligopeptidic derivatives of antracyclines. This adverse reaction was caused by large aggregates of these positively charged oligopeptidic derivatives that likely interact with heparin-like substances lining the surface of blood vessels preventing appropriate anticoagulation. Heparin-like substances can be defined as negatively charged glycosaminoglycan polymers with anticoagulant properties. Negatively charged and neutral prodrugs comprising said molecules were found to overcome this undesirable side effect (WO00/33888). In this way the active drug can be released at the tumor site and taken up by the cancer cells where it implements its intracellular activity. This application is focused on the selective delivery of active drugs at the site of tumors whereby the drug acts intracellularly within the tumor cell. Contrarily, the present invention uses the unique property that these positively charged molecules form aggregates interacting with the surface of the blood vessels to exert a different application. The negative aspect of this feature is turned into a positive aspect by releasing the active compound in the vicinity of the target cell only so that no acute toxicity can arise. The action of the drug is localised extracellularly, preferentially at the neoangiogenic blood vessels.

B is either a covalent assembly of positively charged chemical groups or a positively charged molecule, which in aqueous solutions forms non-covalent polycations due to its propensity to form intermolecular aggregates. According to the present invention, it is possible to induce intra-vascular blood clotting selectively in a tumor's vasculature. The positive charges of covalent or non-covalent polycations inducing intra-vascular clotting through their interaction with heparin-like substances lining endothelia, can be masked with negatively charged or neutral moieties that are sensitive to conditions found selectively in the extracellular milieu of tumors. Therefore the invention provides two preferred embodiments of the compound of the general formula A-B as defined above wherein A and B carry at least one negative charge and at least one positive charge, respectively, whereby the positive charge of B is masked by the negative charge of A and wherein A and B are uncharged moieties in compound A-B, whereby B is able to obtain (a) positive charge(s) when released from A-B.

Compounds according to the invention are non-toxic due to their incapacity to interact with heparin-like substances, but once in the tumor environment, the masking moieties are cleaved and the polycation is regenerated, inducing local coagulation. Therapeutic effects can be achieved by inducing intravascular blood coagulation selectively in capillaries and vessels of solid tumors and their metastases. As a matter of fact, intravascular clotting prevents nutrient and oxygen supply of the cancer cells. Delivering a procoagulant selectively and exclusively to the solid tumors and their metastases will spare the vasculature of normal tissues. The present invention leads to the development of new options for a more efficient treatment of resistant cancers, the major killers. Prodrugs of the invention, i.e. pharmacologically inactive derivatives, can be made of extremely effective procoagulants that would be activated selectively in the environment of tumors, but would be stable in blood and normal tissues. Such prodrugs would be non-toxic and would release the coagulation-promoting agent only within solid tumor tissue and in metastases.

At a minimum, the masking moiety prevents the non-specific degradation of the prodrug and may, as will discussed in more detail below, provide the prodrug with additional favorable properties such as reduced toxicity, increased stability, etc. Preferred masking moieties are those that are stable in vivo, not toxic to healthy cells and not immunogenic.

In view of the prior art, the prodrug described in WO00/33888 comprises i) a stabilizing group, ii) a cleavable oligopeptide, iii) an optional linker and iv) a therapeutic agent. The unmodified therapeutic agent has low solubility and high concentrations and, as mentioned elsewhere, if administered intravenously results in aggregation and intravenous coagulation. The design of the prodrug in WO 00/33888 seeks to:

1) Solubilise the therapeutic agent for intravenous administration and passage through the blood.

2) Inactivate the therapeutic agent during its passage through the blood.

3) Release the therapeutic agent only at the site of the tumor through cleavage at the cleavable polypeptide site by the enzyme Trouase, present extracellularly in target cells.

Some of the critical differences between WO00/33888 and the present invention are

1) The effectively unmodified, active therapeutic agent is non-aggregating and soluble at the target for cellular uptake in WO00/33888 cf the effectively unmodified, active therapeutic agent forms an intentional aggregation at the target in the invention.

2) The said therapeutic agent is taken into the target cell and acts intracellularly to kill the said cell in WO00/33888 cf the invention is not taken into the cell, rather acts intercellularly to induce intravascular blood coagulation and to restrict/stop nutrient and oxygen supply to the target cell.

Futhermore, Calliceti et al (1993) describe combinations of polymer-doxorubicin prodrugs that deliver doxorubicin inside the cell, after which it doxorubicin is cleaved from polymer by hydrolysis. There are clear differences between Calliceti et al (1993) and the present invention: The therapeutic agent acts intracellularly in the former (cf extracellularly in the invention) and the covalent structure of the prodrug in Calliceti et al (1993) is distinguishable from all the embodiments of this invention.

The procoagulant properties of the polycations can be reversibly suppressed by covalently coupling on the free amino groups chemical entities that neutralize or confer a negative charge, and that are known to be unstable at pH 6.0-6.5, conditions that are prevalent in the environment of many tumors. Such masking of the positive charges results in tumor-activated prodrugs of the procoagulant polycation, allowing tumor-selective intravascular blood clotting. Therefore, a preferred embodiment according to present invention is a compound wherein A is a masking moiety carrying a negative charge and is unstable at pH 6.0-6.5. The N-capping group is self-immolative and is chemically removed in the tumor microenvironment thereby releasing active moiety B. In addition, said pH unstable capping moiety can be chosen from the group comprising citraconyl and dimethylmaleyl or combinations thereof or other such moieties well known to those skilled in the art.

Alternatively, the procoagulant properties of the polycations can also be reversibly suppressed by covalently coupling on the free amino groups, and through a linker that can be selectively cleaved in the extracellular tumor environment, chemical entities that neutralize or confer a negative charge but that are not necessarily pH-sensitive. Such masking of the positive charges results in tumor-activated prodrugs of the procoagulant polycation, allowing tumor-selective intravascular blood clotting. In this respect, the present invention further provides a compound as described above wherein A is of the general formula X—Y, wherein X is a neutral or preferably a negatively charged N-capping moiety and Y is a linker stable in normal tissues and body fluids, but that is specifically degraded in the environment of tumors. The group X acts in providing preferable charge to the prodrug, and may also block degradation of the prodrug by exopeptidases or provide other desirable physical prodrug characteristics.

Preferentially, the present invention describes a compound, wherein the N-capping moiety is chosen from the group comprising methyl, succinyl, glutaryl, maleyl, and diglycolyl, polyalkyleneglycols or combinations thereof. These groups are neutral or carry a negative charge and thereby neutralise the positive chemical group(s) located within B. In this case B can only be activated by the cleavage at Y which physically removes the protecting group.

According to present invention said polyalkyleneglycol may be a polyethylene glycol having an average molecular weight ranging from 100 up to 12000 Da; preferably, said polyethylene glycol has an average molecular weight of 350 Da.

Since the masking moiety is linked to the linking moiety, the masking moiety should have a reactive group that is complementary to a reactive group on the linking moiety. For instance, if the linking moiety is a peptide and the masking moiety is to be linked to the amino terminus of the linking peptide, then a free carboxyl group of the masking moiety complements the amino terminus of the linking peptide such that the masking moiety can form an amide bond with the amino terminus of the peptide. Alternatively, the masking moiety should be capable of modification to have a reactive group that complements a reactive group of the linking moiety. The masking moiety can be linked to the linking moiety directly or via a spacer moiety. Those of skill in the art will recognize that, while in most instances the masking moiety and the biological active agent will be linked directly to the termini of the linking moiety, in some instances it may be desirable to space the linking moiety away from either or both masking moiety and biological active agent with a spacing moiety. The linking moiety is linked to the biologically active agent and masking moiety via an amide linkage. However, it will be recognized that virtually any linkage that is stable to the conditions of use (e.g., stable in blood or serum) and that can be readily formed without denaturing or otherwise degrading the masking moiety and/or biologically active agent may be employed. Thus, the masking moiety and biologically active agent may include virtually any reactive group that is complementary to, i.e. able to covalently react with, the respective terminus of the linking moiety to which it will b attached. Suitable groups complementary to the linking moiety amino terminus include, for example, carboxy groups, esters (including activated esters such as NHS-esters), acyl azides, acyl halides, acyl nitriles, aldehydes, alkyl sulfonyl halides, halotriazines, imidoesters, isocyanates, isothiocyanates, sulfonate esters, etc. Suitable reactive groups complementary to the linking moiety carboxy terminus include, for example, amines, alcohols, alkyl halides, thiols, hydrazines, diazoalkanes, sulfonate esters, etc. Conditions for forming covalent linkages between a plethora of complementary reactive group pairs are well known. Preferably, each linkage between the linking moiety and the biologically active agent and the masking moiety is an amide. Conditions for linking molecules together having complementary amino and carboxy groups to form amide linkages are well-known (see, e.g., Merrifield, 1997). Specific linking chemistries are provided in the Examples section. The present invention describes further a compound as described above, wherein Y is a substrate for extracellular tumor hydrolases and that is resistant to hydrolases found in normal tissues and body fluids. Release of B at the tumor site only guarantees a low in vivo toxicity of the therapeutic compound. In addition, based on this feature, the prodrug can be administered at a completely different site than where the tumor is located and at a much higher dose. Hydrolases are defined to be enzymes carrying an activity where cleavage of chemical bonds is involved. These chemical bonds can be located in different kinds of molecules such as sugars, lipids or peptides.

Tumor cells and endothelial cells involved in tumor neoangiogenesis are known to secrete a significantly higher concentration of certain peptidases than normal cells do. Therefore, the present invention describes a compound as described above wherein the hydrolase is preferentially an extracellular tumor peptidase. The structures of the oligopeptide Y are selected to limit cleavage of the oligopeptide by enzymes other than those that may be present in tumor micro-environment. The amino acid sequence of the oligopeptide further ensures specificity for an unknown peptidase released by tumor cells and endothelial cells involved in tumor neoangiogenesis, able to cleave N-□-Ala-Leu-Ala-Leu-doxorubicin into Leu-doxorubicin, and referred to as “enzyme X”. Since normal cells liberate little to no enzyme X in vivo, the compound according to the invention is maintained inactive and/or does not form polycations outside of the tumor region. This oligo peptide, N-□-Ala-Leu-Ala-Leu is a specific substrate for one of the tumor peptidases. Nevertheless, other enzymes recognizing other substrates that can be considered for the activation of prodrugs with different Y oligopeptides include plasmin, plasminogen activators, cathepsin B, cathepsin D, and matrix metalloproteases. Specificity will be guaranteed by choosing the specific peptidic substrate for the respective enzyme.

The compound or prodrug according to present invention is administered to the patient, carried through the bloodstream in a stable form, and when in the vicinity of a target cell, is acted upon by enzyme X. Since the enzyme is only minimally present within the extracellular vicinity of normal cells, the prodrug is maintained and its active portion has only minimal effects on the blood vessels of normal tissues. Thus toxicity to normal tissues is minimised. In the vicinity of target cells, however, the presence of the relevant enzyme in the local environment causes cleavage of the prodrug. Once the X-moiety is eliminated through cleavage of the oligopeptide Y the pharmacologically active moiety gets released. B polycations are released or formed inducing coagulation and blocking the tumor blood vessels, resulting in the starvation of tumor tissue. ‘Normal cells’ means non-target cells that would be encountered by the prodrug upon administration of the prodrug in the manner appropriate for its intended use.

The linking moiety can comprise any molecule that is susceptible to cleavage at or near a target cell. The linking moiety is covalently linked to the masking moiety and to the biologically active agent thereby linking the two. Preferred linking moieties are peptides that are susceptible to cleavage at or near target cells. For example, it has been discovered that peptides having the amino acid sequence (Leu)_{y}(Ala-Leu)ₓAla-Leu and peptides having the amino acids sequence (Leu)_{y}(Ala-Leu)ₓAla-Phe (wherein y=0 or 1 and x=1, 2, or 3) are cleaved by a factor in the extracellular milieu near target cells. Preferred peptide linking moieties comprise amino acid sequence Ala-Leu-Ala-Leu, Leu-Ala-Leu-Ala-Leu, Leu-Ala-Leu, or Leu-Ala. The oligopeptide has a formula or sequence (AA)ₘ-AA⁴-AA³-AA²-AA¹, wherein each AA independently represents any genetically encoded amino acid; m is an integer from 0 to 12; AA⁴ represents a non-genetically-encoded amino acid (called the blocking amino acid); AA³ AA² and AA¹ represents any amino acid (WO00/33888). The function of the blocking amino acid at this position is to maintain selectivity for cleavage of the prodrug by “enzyme X”; decreasing its vulnerability to exopeptidases and endopeptidases present in blood and normal tissues. One preferred amino acid at that position is an □-amino acid, eg. □-Ala. Other possibilities are mentioned in WO00/33888 which application is hereby incorporated by reference.

Preferentially, the present invention describes a compound as described above X—Y—B or as described below X—Y—U—V, wherein Y consists of Y′-L-Leu-L-Ala-Y″ and Y′ and Y″ are amino acids or oligopeptides preferably consisting essentially of L-amino acids; Y′ comprising a blocking amino acid at position AA⁴. It has been shown that the tumor specific peptidase “enzyme X” recognizes this specific peptide sequence. Consequently, an oligopeptide carrying such a Leu-Ala dipeptide gets processed resulting in the release of X—Y′L-Leu and L-Ala-Y″-B. The N-capped tetrapeptide N-succinyl-□-Ala-L-Leu-L-Ala-L-Leu was found to be an optimal substrate for this “enzyme X” (WO00/33888). Therefore the present invention describes a compound as described above wherein Y is preferentially □-Ala-L-Leu-L-Ala-L-Leu and X a succinyl group. Released L-Ala-Y″-B contains the active compound B and can be further processed by exopeptidases resulting in the Y″-B intermediate (L-Leu-B). Since these derivatives are positively charged, cleavage by “enzyme X” restores the pro-coagulant activity. Alternatively, as discussed before other groups than succinyl might be used.

WO00/33888 contains embodiments which allow for a combination of elements in the prodrug and those which might be considered to be chemically the most similar to this invention are:

Suc-□-Ala-Leu-Ala-Leu-Dox (claim 66)

Suc-□-Ala-Leu-Ala-Leu-Dnr (claim 66)

Glutaryl-□-Ala-Leu-Ala-Leu-Dox (claim 66)

Suc-□-Ala-Leu-Ala-Leu (claim 67)

The N-capped tetrapeptide N-succinyl-□-Ala-L-Leu-L-Ala-L-Leu is also contained within the construction of the prodrug described in this invention (p8 para. 2). However, as discussed above, the said prodrug construction contains different or additional covalently attached elements, which, on examining all their possible permutations do not combine produce any molecules matching those of WO00/33888.

According to the present invention, moiety B is either a covalent assembly of positively charged chemical groups or a positively charged molecule which in aqueous solutions is able to form non-covalent positively charged polymer-like assemblies. Nevertheless, it does not exclude the possibility that these covalent assemblies may aggregate and form complex non-covalent assemblies. Eg. poly-D-Lys coupled to a hydrophobic structure can still form aggregates before binding to heparin-like substances. With polymer is meant a molecule comprising at least 2 covalently coupled molecules. These molecules may have any structure eg. organic molecule, lipid, nucleotide, amino acid or combinations thereof.

Positively charged covalent polymers can be used as the procoagulant moiety provided they are modified as described. Otherwise, they would be too toxic to be used as therapeutics.

The present invention describes further a compound as described above, whereby the moiety B comprises a peptide consisting essentially of D-amino acids or derivatives thereof. The use of D-amino acids stabilizes the polycation once it is released from the complex prodrug structure with regard to the action of peptidases present in body fluids and tissues. This guarantees a long acting character of the molecule.

Preferably, a compound according to the present invention as described above, wherein said B moiety comprises poly-D-Lys. Poly-D-Lys interacts with heparin-like substances lining all vascular structures, thereby inducing coagulation. These polymers are highly toxic when injected into animals as such due to their non-specific interaction with both tumor and normal tissues endothelial cells. But when incorporated into a prodrug structure as described by the present invention, a non-toxic and tumor specific killer product is formed. Also poly-D-Arg can be used (Carr et al., 1989).

Preferably, a compound according to the present invention as described above, wherein said B moiety comprises a D-amino acid version of heparin-binding peptides such as (AKKARA)ₙ or (ARKKAAKA)ₙ (Verrechio et al., 2000); n is an integer from 1 to 10. Heparin-binding peptides can be defined as peptides having an affinity for heparin-like substances and whose interactions with heparin-like substances inhibit its anticoagulant properties. Heparin-binding peptides may carry different affinities for heparin. Peptides with low affinity for heparin may form aggregates resulting in high affinity complexes. Similarly to the poly-Lys, once these peptides are incorporated into a prodrug as defined by the present invention, a non-toxic and tumor specific killer product is formed.

Preferably, the present invention is concerned with compounds as described above wherein amino-groups of said procoagulant moiety B are branched with at least one D-amino acid. To enhance the procoagulant properties of the polymer, side-chains can be branched with D-amino acids or oligopeptides made of D-amino acids. Coupling of amino acids or oligopeptides through their carboxy-terminus increases the volume of the positively-charged “particle”, resulting in improved activity. D-Amino acids are preferred because they allow a good in vivo stability of the compound as described above.

Instead of a synthetic positively-charged polymer, molecules that have a natural tendency to stack, pile up and aggregate, and to which peptides can be hooked through their carboxy-terminus can be used. The aggregates (“non-covalent” polymers) formed by the peptidic conjugates of those molecules will similarly induce intravascular blood clotting. In case the molecule comprises an amino acid(s) D-amino acids must be preferably used for stability reasons as described above. The procoagulant activity can be reversibly masked just like in the case of the covalent polycatons using pH- or tumor peptidase-sensitive neutral or negatively-charged moieties. In particular, the present invention relates to a compound as defined above whereby the procoagulant moiety B is of the general formula U—V, wherein U is an oligopeptide consisting essentially of D-amino acids carrying a positive charge and V is a hydrophobic aggregation inducer stable in normal and tumor body fluids and to which U can be covalently hooked through its terminal carboxyl group without interfering with, and reinforcing the aggregation properties of V. Hydrophobic residues tend to make hydrophobic packages by stacking different compounds together. This stacking effect results in the formation of aggregates whose size depends on the concentration of the compound and on the chemical structure of the compound itself. Anthracyclines (Chaires et al., 1982; Menozzi et al., 1984) or other compounds such as: acridine dyes (Li and Crothers, 1969; Müller and Crothers, 1975), purine and pyrimidine derivatives (Dimicoli and Hélène 1973; Tribolet and Sigel, 1987a; Tribolet and Sigel, 1987b; Plesiewicz et al., 1976), dihydroantraquinones (Kharasch and Novak, 1984) and nicotinamide (Coffman and Kildsig, 1996; Charman et al., 1991) can be used to build the procoagulant moiety (FIG. 4).

The inventors observed that oligopeptidic derivatives of anthracyclines, made mainly of hydrophobic amino acids and presenting one or more free amino groups, form much larger, positively-charged aggregates in solution in buffer, serum or blood (WO00/33888). The size of the aggregates can reach several thousand kilodaltons, but is concentration-dependent. After intravenous injection to mice or rats, these anthracycline derivatives were extremely toxic and killed animals in a few minutes at dose levels as low as 35 μmol/Kg. The toxicity symptoms and histopathological results indicated that they extremely rapidly induce massive intravascular coagulation. This intravascular coagulation is very likely the result of the interaction of the polycationic aggregates with the heparin-like (negatively charged) substances that cover the luminal surface of the endothelium of all blood vessels, thereby inhibiting their anticoagulant properties (Cofrancesco et al., 1980; DeLucia III et al., 1993). Inventors also proved that the toxicity of these anthracycline-oligopeptide conjugates could be prevented by prior or simultaneous administration of heparin, which confirms this hypothesis (WO00/33888). In addition, the toxic effects were also completely abolished when the free amino groups of the peptides were capped with neutral or negatively charged groups.

The present invention may also relate to a compound carrying the same properties as described above but in a reverse order. The invention provides therefore a compound A-B as described above whereby the procoagulant moiety B is of the general formula U—V, wherein U is an oligopeptide consisting essentially of D-amino acids carrying hydrophobic residues and V is a positively charged moiety which is covalently bound to U via its terminal amino group. Another embodiment is available according to the invention in which the procoagulant moiety B is of the general formula U—V, wherein U is an oligopeptide consisting essentially of D-amino acids covalently bound to V through its terminal carboxyl group, whereby both U and V carry positive charges and allowing intermolecular interactions.

Preferentially, said D-amino acids are chosen from the group comprising D-Ala, D-Val, D-Leu, D-Ile, D-Phe, D-Trp, D-Cys, D-Asn, D-Gln, D-Ser, D-Thr, D-Tyr, D-Lys and D-Arg.

Preferably, a compound according to the present invention with formula A-U—V or X—Y—U—V carries a tetrapeptide of D-amino acids in its U position. Tetrapeptides are sufficient to prevent the intracellular penetration of small molecules. Indeed, the activity of the molecules described is located outside the cell and no penetration is needed or even desired. Consequently, the peptide should be at least four amino acids but can contain up to eight residues.

Preferably, said U-tetrapeptide is chosen from the group comprising N-D-Ala-D-Leu-D-Ala-D-Leu and N-D-Ala-D-Lys-D-Ala-D-Leu. These tetrapeptides work when V is doxorubucin. Indeed, the present inventors showed that N-D-Ala-D-Leu-D-Ala-D-Leu-doxorubicin or -daunorubicin induces disseminated intravascular blood clotting after intravenous administration at dose levels as low as 34.5 μmol/kg.

The prodrug is administered to the patient and when in the vicinity of a target cell, is acted upon by enzyme X. Cleavage can result in incomplete processing of A-B molecules. So, compounds can be formed containing one or more additional amino acid residues at their N-terminal end. These incompletely processed molecules still act as active anti-tumor compounds according to present invention as described above. When the masking moiety is a N-capped peptide sensitive to the action of tumor-released peptidases, it is not an absolute requirement that the peptidase cleaves exactly between the linking peptide (moiety Y) and the procoagulant (moiety B). The presence of one or two extra L-amino acids onto the B moiety does not affect its activity since it is not affecting its positive charge.

The present invention also relates to a compound as described above for use as a medicine. The use of the described compounds should result in intravascular blood clotting, affecting selectively tumor vasculature. As a result of nutrient and oxygen deprivation, tumor growth should be prevented and tumor regressions could also occur. Therefore, this approach could be a potent cancer therapy.

The present invention also relates to a compound as described above for the manufacture of a medicament for the treatment and/or prevention of tumor related disorders. The term ‘tumor related disorder’ refers to an abnormality within a patient or animal where normal cells start to proliferate due to a change in the coding or non-coding sequence of the DNA resulting in a swollen or distended tissue. An abnormal malignant mass of tissue is formed that is not inflammatory, and from which metastatic cells spread throughout the body to form secondary tumors that eventually cause the death of the patient. Cells of pre-existent tissue start to divide unexpectedly and resulting cell mass possesses no physiologic function. Due to the mass of cells, which results from the high division rate of the cells, a lot of oxygen is needed. If blood coagulation could be induced in tumor vasculature, sparing the normal vessels, it would be possible to starve tumors, and therefore to block their growth or to induce their disappearance.

The antitumoral therapy may be repeated intermittently while tumors are detectable or even when they are not detectable. The therapy may be provided alone or in combination with other drugs, such as for example other antiangiogenic agents, antineoplastic agents or other pharmaceutically effective agents.

The pharmaceutical compositions comprising the prodrugs of the invention may be manufactured by means of conventional mixing, dissolving, emulsifying, or lyophilizing processes. Pharmaceutical compositions may be formulated in conventional manner using one or more physiologically acceptable carriers, diluents, excipients or auxiliaries which facilitate processing of the active peptides into preparations which can be used pharmaceutically.

The pharmaceutical composition may be administered to the patient parenterally, subcutaneously, intrathecally, intramuscularly or intraperitoneally, preferentially intravenously. Pharmaceutical compositions of the invention for intravenous administration comprise sterile, aqueous or nonaqueous solutions or emulsions. As a pharmaceutically acceptable solvent or vehicle, propylene glycol, polyethylene glycol, injectable organic esters, for example ethyl oleate, or cyclodextrins may be employed. These compositions can also comprise wetting, emulsifying and/or dispersing agents.

For injection, the prodrugs of the invention may be formulated in aqueous solutions, preferably in physiologically compatible buffers such as Hank's solution, Ringer's solution, or physiological saline buffer. The solution may contain formulatory agents such as suspending, stabilising and/or dispersing agents.

Alternatively, the prodrug may be in powder form for reconstitution with a suitable vehicle, e.g., sterile pyrogen-free water, before use.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

As the prodrugs of the invention may contain charged side chains, they may be included in any of the above-described formulations as the free acids or as pharmaceutically acceptable salts. Pharmaceutical acceptable salts are those salts which substantially retain the activity of the free acids and which are prepared by reaction with inorganic bases. Pharmaceutical salts tend to be more soluble in aqueous and other protic solvents than are the corresponding free acid forms.

The sterilisation may be carried out in several ways, for example using a bacteriological filter, by incorporating sterilising agents in the compositions or by irradiation. The prodrugs of the invention may also be prepared in the form of sterile solid compositions, which may be dissolved at the time of use in sterile water or any other injectable medium.

The pharmaceutical composition may also comprise adjuvants, which are well known in the art (e.g., vitamin C, antioxidant agents, etc.) and capable of being used in combination with the compound of the invention in order to improve and prolong the treatment of the medical condition for which they are administered.

The prodrugs of the invention can be used in a wide variety of applications to inhibit or prevent the growth of a tumor. For example, the prodrugs can be used to treat or prevent diseases related to tumor cell growth in animals. In addition, human patients are the usual recipients of the prodrug of the invention, although veterinary usage is also contemplated.

The present invention also relates to a method for the treatment of a subject in need of an anti-tumor treatment administering a therapeutically effective amount of a compound according the present invention as described above. It is to be understood that the amount used will depend on the particular application.

For example, for use as an anti-angiogenic agent, a therapeutically effective amount of a prodrug, or composition thereof, is applied or administered to an animal or human in need thereof. By therapeutically effective amount is meant an amount of prodrug or composition that ameliorate the symptoms, or ameliorate, treat or prevent tumor growth or diseases related thereto; more specifically, it induces blood clotting at the tumor sites only resulting in the disruption of the tumor vascularisation and consequently in the control of tumor growth. An ordinarily skilled artisan will be able to determine therapeutically effective amounts of particular prodrugs for particular applications without undue examination using, for example, the in vivo assays provided in the examples.

Initial dosages can be estimated from in vivo data, e.g., animal models, using techniques that are well known in the art. One having ordinary skill in the art could readily optimise administration to humans based on animal data. Dosage amount and interval may be adjusted individually to provide plasma levels of the prodrug which are sufficient to maintain therapeutic effect. Therapeutically effective serum levels may be achieved by administering multiple doses each day. The amount of prodrug administered will, of course, be dependent on the subject being treated, on the subject's weight, the severity of the affliction, the manner of administration and the judgement of the prescribing physician.

Preferably, a therapeutic effective dose of the prodrugs described herein will provide therapeutic benefit without causing substantial toxcity.

Toxicity of the prodrugs described herin can be determined by standard pharmaceutical procedures in experimental animals, e.g., by determining the LD₅₀ (the dose lethal to 50% of the population) or the LD₁₀₀ (the dose lethal to 100% of the population). The dose ratio between therapeutic and toxic effect is the therapeutic index. Compounds which exhibit high therapeutic indices are preferred. The dosage of the prodrugs described herein lies preferentially within a range of circulating concentrations that include the effective dose with little or no toxicity. The dosage may vary within this range upon the dosage form. The exact formulation and dosage can be chosen by the individual physician in view of the patient's condition (see e.g., Fingl et al. 1975).

In a further aspect the invention is related to a method for synthesizing a compound A-B comprising the step of reacting a precursor of A with a precursor of B under conditions in which a reactive group of A condenses with a complementary reactive group of B, thereby forming A-B,

or

1) reacting a precursor of X with a precursor of Y* under conditions in which a reactive group of X condenses with a complementary reactive group of Y*, thereby forming X—Y*, whereby Y* is a precursor of Y comprising a reactive group*, and,

2) reacting X—Y* with a precursor of B under conditions in which a reactive group of X—Y* condenses with a complementary reactive group of the precursor of B thereby forming X—Y—B,

or

1) reacting a precursor of *U with a precursor of V under conditions in which a reactive group of *U condenses with a complementary reactive group of V, thereby forming *U—V, whereby *U is a precursor of U comprising a reactive group*, and,

2) reacting *U—V with a precursor of A under conditions in which a reactive group of *U—V condenses with a complementary reactive group of the precursor of A thereby forming A-U—V,

or

1) reacting a precursor of X with a precursor of Y* under conditions in which a reactive group of X condenses with a complementary reactive group of Y*, thereby forming X—Y*, whereby Y* is a precursor of Y comprising a reactive group *, and,

2) reacting a precursor of *U with a precursor of V under conditions in which a reactive group of *U condenses with a complementary reactive group of V, thereby forming *U—V, whereby *U is a precursor of U comprising a reactive group*, and,

3) reacting X—Y* with *U—V under conditions in which a reactive group of X—Y* condenses with a complementary reactive group of *U—V thereby forming X—Y—U—V.

The following definitions serve to illustrate the terms and expressions used in the present invention.

“Biologically active agent” refers to a positively charged molecule or construct that promotes blood coagulation.

“Linking moiety” refers to a molecular moiety that is capable of linking a biologically active agent to a masking moiety. A linking moiety is susceptible to specific, selective cleavage at or near a tumor site or cell. A linking moiety possesses a reactive group that complements a reactive group on a biologically active agent and a second reactive group that complements a reactive group in a masking moiety such that the linking moiety is capable of linking the biologically active agent to the masking moiety. The reactive group of the biologically active agent and/or the reactive group of the masking moiety can be added to the agent and/or masking moiety by modification if necessary.

“Masking moiety” refers to a molecular moiety that, when linked to a biologically active agent via a linker moiety, is capable of masking the biological activity of the agent and is capable of preventing the non-specific degradation of the linker moiety.

“Selective cleavage” refers to cleavage at or near a tumor or target cell. Selective cleavage is not necessarily unique to the environment at or near a tumor or target cell. Rather, selective cleavage refers to enriched or preferentially cleavage at or near a tumor or target cell.

“Specific cleavage” refers to cleavage of a bond within the linking moiety or of the bond between a linking moiety and a biologically active agent.

“Heparin-like substances” can be defined as negatively charged glycosaminoglycan polymers with anticoagulant properties.

“Heparin-binding peptides” can be defined as peptides having an affinity for heparin-like substances and whose interactions with heparin-like substances inhibit its anticoagulant properties.

To avoid confusion with the various formulae used herein, genetically-encoded amino acid residues are exclusively designated with the three letter abbreviations. The conventional three letter code is used as known by a person skilled in the art.

The following examples and figure legends merely serve to illustrate the invention and are by no way to be understood as limiting the present invention.

### FIGURE LEGENDS

FIG. 1. Structural formula of daunorubicin.HCl.

FIG. 2: Retrosynthesis of a PEGylated prodrug of a procoagulant entity.

FIG. 3: PEG activation and peptide coupling.

FIG. 4: Candidate aggregation inducers whose derivatives could be included in procoagulant peptidic conjugates.

FIG. 5: Poly-Lys-based procoagulants and their prodrugs.

FIG. 6: Retrosynthesis of a tumor-activated prodrug of a procoagulant DNR derivative.

### MODES FOR CARRYING OUT THE INVENTION

The present invention describes (a) compound(s) that can be used to induce intravascular coagulation restricted to the capillaries of solid tumors and their metastases thereby preventing nutrient and oxygen supply of the cancer cells. Prodrugs are made and active compounds are released at the site of the tumor. For selective activation of these prodrugs the inventors have taken the advantage of the oversecretion of certain peptidases by tumor cells and by endothelial cells involved in tumor neoangiogenesis (Werb et al., 1999). In addition, the inventors have confirmed the “proof of principle” of this new concept based on the procoagulation properties of a tetrapeptide derivative of daunorubicin (DNR). DNR was selected because it is a good model substance that can be easily linked to peptides, and because the inventors previously analysed the procoagulation properties of its oligopeptidic derivatives (WO00/33888). The concept was proven based on such derivatives and the inventors have extended it to compounds based on non-cytotoxic aggregation-inducing small molecules to replace the anthracycline, as well as to polycationic polymers.

### EXAMPLE 1

[1] Stable procoagulant peptidic derivative of DNR.

The inventors have prepared tetrapeptidic derivatives of DNR. In order to obtain derivatives that remain stable in the blood and are not reactivated into DNR by tumor cells D-amino acids were used. Therefore D-Ala-D-Leu-D-Ala-D-Leu-DNR was the first compound that was prepared.

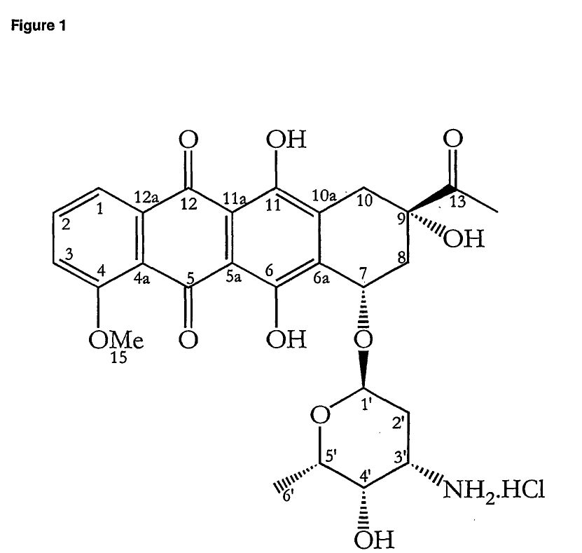

FIG. 1 shows the structure of DNR. N-Fmoc-D-Ala-D-Leu-D-Ala-D-Leu-OH, prepared by classical solid phase peptide chemistry (Pennington et al., 1994), was coupled to the free amino group of the daunosamine moiety using 2-(1H-9-azabenzotriazole-1-yl)-1,1,3,3-tetramethyluronium hexafluorophosphate (HATU) as the coupling reagent in dimethylformamide (DMF) in the presence of diisopropylethylamine (DIPEA). N-Fmoc-D-Ala-D-Leu-D-Ala-D-Leu-DNR was isolated by water precipitation before deprotection of the N-terminus using piperidine in DMF. Subsequently, the lactate salt of N-D-Ala-D-Leu-D-Ala-D-Leu-DNR was prepared.

[2] Toxicity/procoagulant activity of N-D-Ala-D-Leu-D-Ala-D-Leu-DNR

N-D-Ala-D-Leu-D-Ala-D-Leu-DNR was injected in the lateral tail vein of OF-1 mice at different dose levels in order to determine the minimal dose that induces acute toxicity and kills the animals in less than an hour following the injection. Animals were injected with this dose level and sacrificed befor death resulting from the injection of the conjugate occurs. Organs and tissues were collected, fixed and prepared for histopathology.

[3] Tumor-selective prodrugs of N-D-Ala-D-Leu-D-Ala-D-Leu-DNR

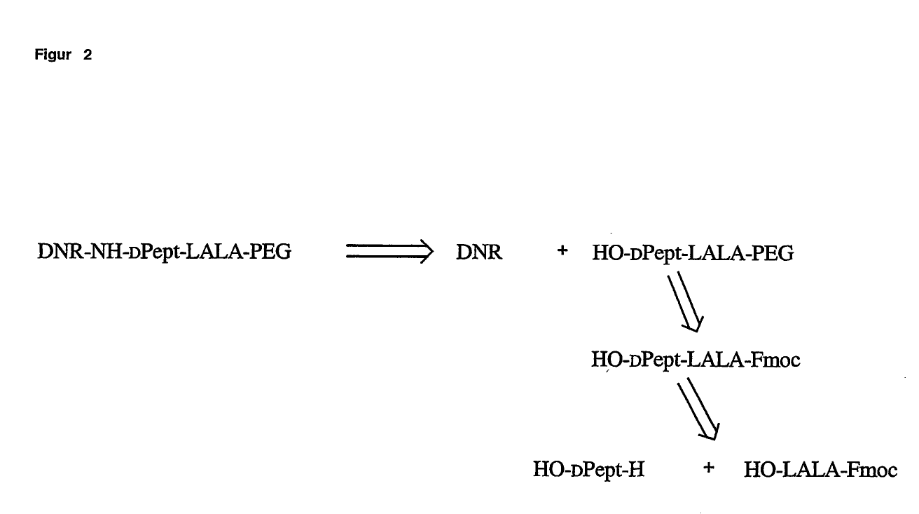

A tumor-activated prodrug of this procoagulant DNR derivative was synthesized by linking an □-Ala-L-Leu-L-Ala-L-Leu peptide N-capped with methoxypoly(ethylene)glycol (mPEG) 350 to the amino terminus of the DNR-D-tetrapeptide. The retrosynthesis of the corresponding prodrug of the mPEG-□-Ala-L-Leu-L-Ala-L-Leu-D-Ala-D-Lys-D-Ala-D-Leu-DNR procoagulant moiety is illustrated in FIG. 2. Polyethylene glycol is a polymer that has been frequently conjugated to peptides, proteins or drugs to reduce their elimination rate and/or to increase their solubility. Here it was mainly used to mask the terminal positive charge and to stabilize the peptide with regard to the action of blood peptidases and cleavage by peptidases. Monoprotected PEG was used. The free hydroxyl group was activated prior to coupling to the octapeptide. The PEGylated octapeptide was then coupled to DNR using HATU as described in point # 1 of the ‘Example 1’, Different types of bonds are possible between mPEG and the peptide, but preferably a strong carbamate linkage.

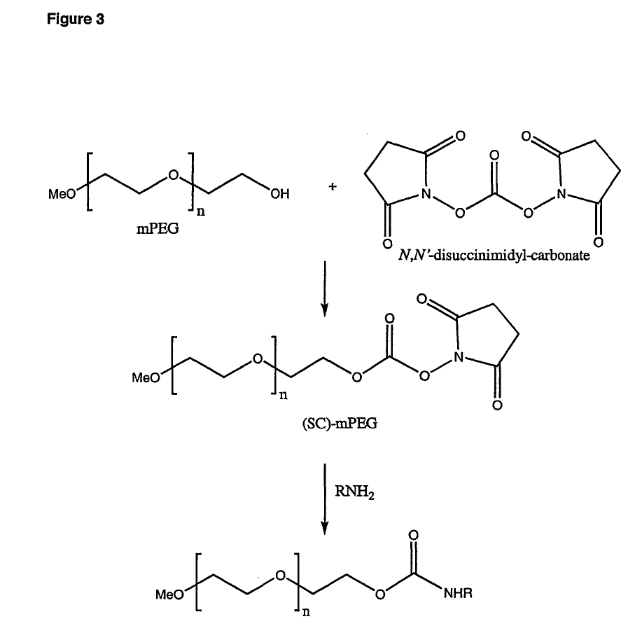

Fmoc-□-Ala-Leu-Ala-Leu-D-Ala-D-Leu-D-Ala-D-Leu-OH was also prepared by classical solid phase peptide synthesis. It was N-capped with mPEG350 previously activated with disuccinimidylcarbonate. The synthesis of this mPEG-peptide conjugate is illustrated in FIG. 3. The N-hydroxysuccinimide ester of the resulting mPEG-tetrapeptide was then prepared in presence of N-hydroxysuccinimide and a carbodiimide, and reacted with the DNR.

The stability of this prodrug in whole human blood was determined, and its activation into the DNR-D-tetrapeptide (or any other positively charged derivative) was checked after incubation in the presence of human cancer cells conditioned media. Breast (MCF-7, MDA-MB-231), colon (LS-174-T), prostate (LNCaP) and lung (COR-L23, NCl-H69) cancer cell lines were used. Metabolites were analyzed by reverse phase-high pressure liquid chromatography (RP-HPLC), and fluorescence detection was used to identify and quantify the DNR derivatives generated over time. The prodrug was relatively stable in blood and yielded the positively-charged N-L-Leu-D-Ala-D-Leu-D-Ala-D-Leu-DNR derivative upon incubation in cancer cells conditioned media. This particular prodrug is suitable for the in vivo validation of the concept.

[4] Toxicity of N-mPEG350-□-Ala-L-Leu-L-Ala-L-Leu-D-Ala-D-Leu-D-Ala-D-Leu-DNR

The acute toxicity of the model prodrug was studied and its LD₅₀ determined 14 and 28 days after intravenous injection to OF-1 normal mice from cumulative mortality curves. Single and multiple injection protocols were tested.

[5] Chemotherapeutic activity of N-mPEG350-□-Ala-L-Leu-L-Ala-L-Leu-D-Ala-D-Leu-D-Ala-D-Leu-DNR.

The prodrug of the procoagulant peptide conjugate of DNR was evaluated in different experimental tumor models. Subcutaneous tumors obtained from the cell lines mentioned in point # 3 were grown in athymic mice. Once tumors have reached a mean diameter of at least 6 mm, the mice were treated with either saline or increasing dose levels of the prodrug once a week for 6 to 8 weeks. The evaluation was performed at dose levels as close as possible to the maximal tolerated dose. Efficacy was evaluated based on the reduction of tumor growth in treated groups as compared to controls. Toxicity was assessed based on clinical signs and the evolution of body weights.

[6] Tissue distribution studies

Tissue distribution studies were performed in tumor-bearing mice and confirmed the mode of action, i.e. the generation of a positively-charged procoagulant metabolite specifically within the tumor. Mice bearing the same tumor types used for efficacy studies were used. They were injected i.v. with the prodrug, and anesthetized at selected time points. Plasma and tissues (tumor, liver, kidney, spleen, heart, lung and skeletal muscle) were recovered, snap frozen in liquid nitrogen and stored at −80° C. The prodrug and its metabolites were then extracted and quantified by RP-HPLC with fluorescence detection.

### EXAMPLE 2

Optimization processes resulted in alternative moieties of A and B.

[1] Alternative procoagulant moieties

Other D-tetrapeptide can also be coupled to DNR in order to produce an appropriate procoagulant moiety. D-Ala-D-Lys-D-Ala-D-Leu is one such peptide.

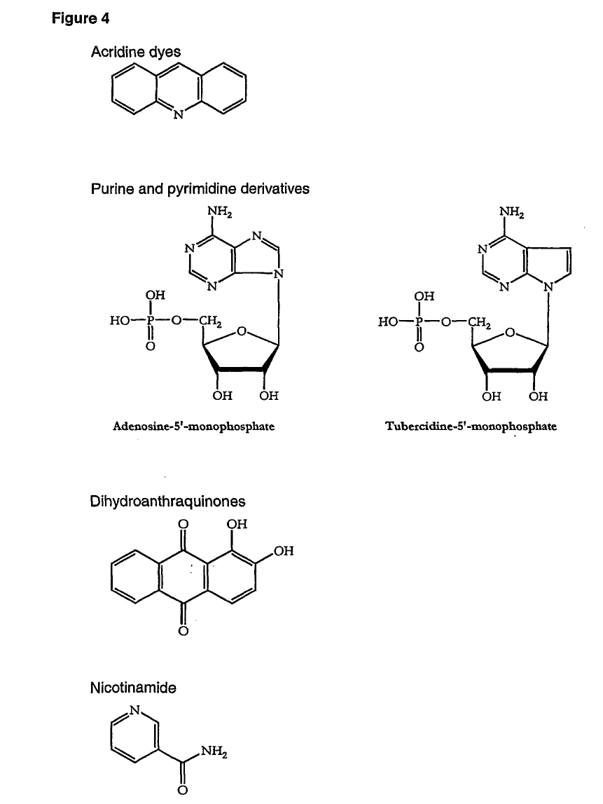

The inventors have also screened non-cytotoxic aggregation inducers to replace DNR. Although the free, active anthracycline is not supposed to be released in vivo, the use of a non-toxic entity is preferable for the successful and rapid development of such a new technology. Derivatives of compounds such as those presented in FIG. 4 allowing derivatization with oligopeptides (e.g. including a primary amine) were considered. Acridine dyes (Li and Crothers, 1969; Müller and Crothers, 1975), purine and pyrimidine derivatives (Dimicoli and Hélène, 1973; Tribolet and Sigel, 1987a; Tribolet and Sigel, 1987b, Plesiewicz et al., 1976), dihydroanthraquinones (Kharash and Novak, 1984) and nicotinamide (Charman et al., 1991; Coffman and Kildsig, 1996) are known for their tendency to form aggregates in solution.

The D-tetrapeptides were coupled to amino derivatives of these compounds produced by classical organic synthesis methods, and the resulting conjugates were compared to N-D-Ala-D-Leu-D-Ala-D-Leu-DNR with regard to their procoagulant activity (see point # 2 of the ‘Example 1’). New candidates were selected based on the results of these simple assays and were used for the synthesis and evaluation of prodrugs as described above.

Whatever the aggregation inducer used, the sequence of D-amino acids used in the procoagulant entity is not, in certain limits at least, of crucial importance with regard to activity. Therefore, other sequences were also considered, particularly when problems were encountered with the previously mentioned sequence. For example, solubility problems occurred in the course of prodrug syntheses or of evaluation, and these were easily solved by the use of another peptide sequence. A number of different sequences were screened rapidly for their procoagulant activity.

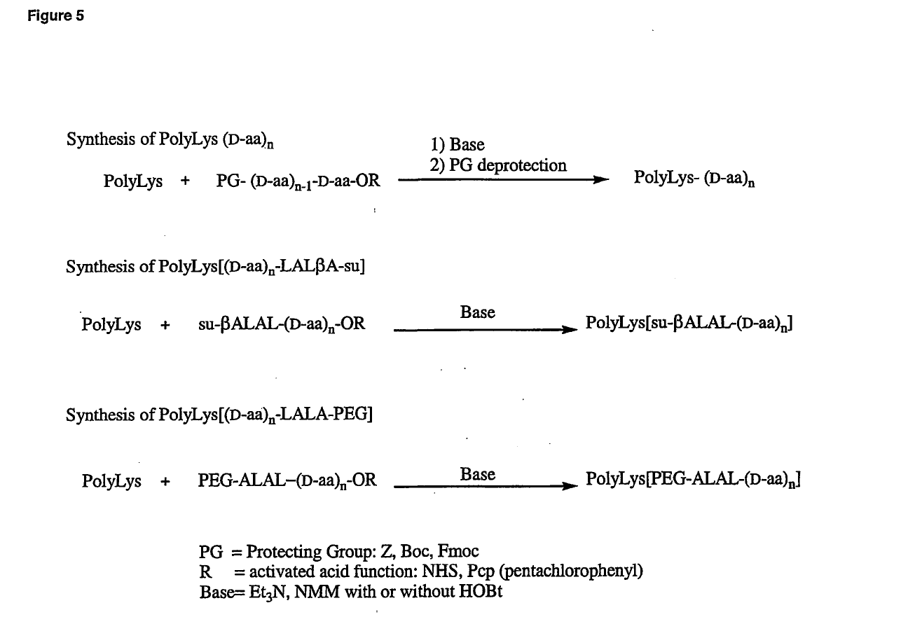

An alternative to aggregation inducers to form the positively charged ‘particles’ responsible for coagulation is to use polymers such as poly-Lys. Poly-D-Lys is available commercially and was positively tested. POIY-D-Lys of different size derivatized with D-amino acids, dipeptides, tripeptides or tetrapeptides on their terminal and side chain free amino groups were prepared (FIG. 5). Their toxicity and procoagulant activities were compared, and interesting candidates were used for the preparation of prodrugs (FIG. 5). N-succinyl-□-Ala-L-Leu-L-Ala-L-Leu or PEG-Ala-Leu-Ala-Leu prodrugs were prepared and compared in terms of efficacy and toxicity to the prodrugs based on aggregation inducers as described above. Alternatively, tests were performed using poly-D-Arg.

[2] Alternative pro-moieties

The inventors have used mPEG350-□-Ala-L-Leu-L-Ala-L-Leu as the initial promoiety to validate the concept. However, other pro-moieties that mask the positive charges of the procoagulant, that are stable in blood and specifically cleaved by peptidases released by tumor cells or endothelial cells involved in tumor angiogenesis do exist and were considered.

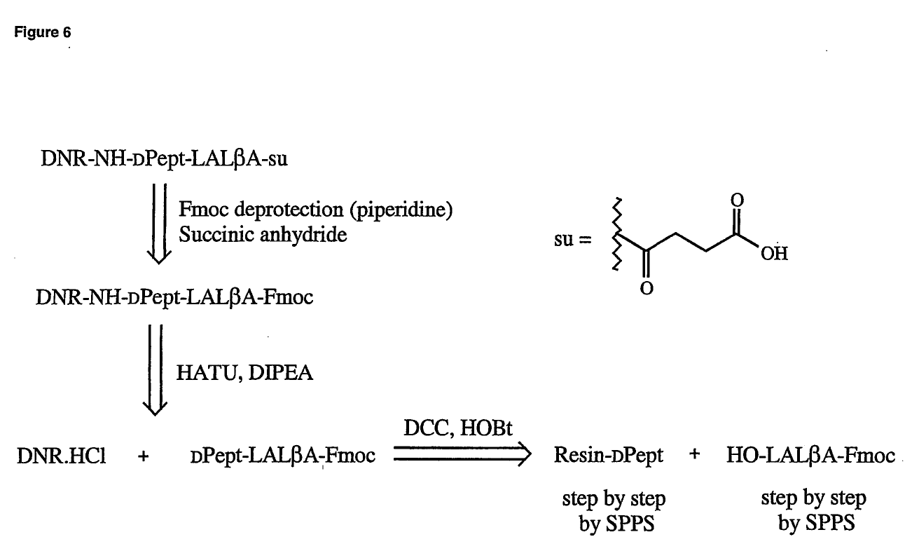

N-succinyl-□-Ala-L-Leu-L-Ala-L-Leu, for example was evaluated. The retrosynthesis of the corresponding prodrug of the D-Ala-D-Lys-D-Ala-D-Leu-DNR procoagulant moiety is illustrated in FIG. 6.

References

Boehm T., Folkman J. and Browder T. et al. 1997. Antiangiogenic therapy of experimental cancer does not induce acquired drug resistance. Nature 390, 404-407.

Calliceti P et al 1993. Preparation and properties of monomethoxy poly(ethylene glycol) doxorubicin conjugates linked by an amino acid or a peptide as spacer. Farmco, Societa Chemica Italiana Pavia, It 48:919-932.

Carr M. E. et al., 1989. Biochemistry 28: 1384-1388.

Chaires J. B. et al., 1982. Biochemistry 21: 3927-3932.

Charman W. N., Lai C. S. and Finnin B. C., et al., 1991. Self-association of nicotinamide in aqueous solution: mass transport, freezing-point depression, and partition coefficient studies. Pharm. Res. 8, 144-1150.

Coffman R. E. and Kildsig D. O. 1996. Self-association of nicotinamide in aqueous solution: light-scattering and vapor pressure osmometry studies. J. Pharm. Sci. 85, 848-853.

Cofrancesco E., Vigo A. and Pogliani E. 1980. Antiheparin activity of adriamycin. Thromb. Res. 18, 743-746.

Colombo T. et al. 1981. Interaction between heparin and adriamycin in mice bearing the lewis lung carcinoma. Biomed. 34:124-128.

Confalonieri C., Cristina G. and Farina M. 1991. The use of a new laser particle sizer and shape analyser to detect and evaluate gelatinous microparticles suspended in reconstituted anthracycline infusion solutions. J. Pharm. Biomed. Anal. 9, 1-8.

Dalmark, M. and Storm H. H. 1981. A Fickian diffusion transport process with features of transport catalysis. Doxorubicin transport in human blood cells. J. Gen. Physiol. 78: 349-364.

DeLucia A. III, Wakefield T. W. and Andrews P. C., et al., 1993. Efficacy and toxicity of differently charged polycationic protamine-like peptides for heparin anticoagulation reversal. J. Vasc. Surg. 18, 49-60.

Dimicoli J. L. and Hélène C. 1973. Complex formation between purine and indole derivatives in aqueous solutions. Proton magnetic resonance studies. J. Am. Chem. Soc. 95,1036-1044.

Ekrami H. M. and Shen W. C. 1995. Carbamylation decreases the toxicity but not the drug-carrier properties of polylysins. J. Drug Targ. 2: 469-475.

Fingl et al. 1975. In: The Pharmacological Basis of Therapeutics, Ch.1, p1.

Fujihira S., Yamamoto T. and Yoshizawa K., et al. 1993. The high incidence of atrial thrombosis in mice given doxorubicin. Toxicol. Pathol. 21, 362-368.

Harris A. L. 1997. Antiangiogenesis for cancer therapy. The Lancet 349, 13-15.

Horrow J. C. 1985. Protamine: a review of its toxicity. Anesth. Analg. 64: 348-361.

Kharash E. D. and Novak R. F. 1984. Spectroscopic evidence for anthracenedione antineoplastic agent self-association and complex formation with flavin nucleotides. Arch. Biochem. Biophys. 234, 497-512.

Koslow A. R. et al. 1987. Protamine-induced pulmonary edema in rats. Current Surg. 44: 219-224.

Li H. J. and Crothers D. M. 1969. Relaxation studies of the proflavine-DNA complex: the kinetics of an intercalation reaction. J. Mol. Biol. 39, 461-477.

Lijnen H. R. et al. 1983. Heparin binding properties of human histidine-rich glycoprotein. J. Biol Chem. 258: 3803-3808.

Lindblad, B. 1989. Protamine Sulphate: a review of its effects: hypersensitivity and toxicity. Eur. J. Vasc. Surg. 3: 195-201.

Menozzi M., Valentini L. and Vannini E. et al. 1984. Self-association of doxorubicin and related compounds in aqueous solution. J. Pharm. Sci. 73, 766-770.

Merrifield, B. 1997. Concept and early development of solid-phase peptide synthesis. Methods Enzymol. 289: 3-13.

Mizuno Y. et al. 1995. Doxorubicin-heparin complex: reduction of cardiotoxicity of doxorubicin. J. Cancer Res. Clin.Oncol. 121: 469-473.

Molema G., Meijer D. K. F. and de Leij L. F. M. H. 1998. Tumor vasculature targeted therapies. Biochem. Pharmacol. 55, 1939-1945.

Müller W. and Crothers D. M. 1975. Interactions of heteroaromatic compounds with nucleic acids, 1. The influence of heteroatoms and polarizability on the base specificity of intercalating ligands. Eur. J. Biochem. 54, 267-277.

Pennington M. W. and Dunn B. M. 1994. Eds. Methods in Molecular Biology vol. 35: Peptide Synthesis. Humana Press, Totowa.

Plesiewicz E., Stepien E. and Bolewska K., et al. 1976.Stacking self-association of pyrimidine nucleosides and of cytosines: effects of methylation and thiolation. Nucl. Acids Res. 3, 1295-1306,

Roelofse J. A. and van der Bijl 1991. An analphylactic reaction to protamine sulphate. Anesth. Prog. 38: 99-100.

Tainsh K. R. et al. 1992. Mast cell heterogeneity in man: unique functional properties of skin mast cells in response to a range of polycationic stimuli. Immunopharmacol. 24:171-180.

Tribolet R. and Sigel H. 1987a. Self-association of adenosine 5′-monophosphate (5′-AMP) as a function of pH and in comparison with adenosine, 2′-AMP and 3′-AMP. Biophys. Chem. 27,119-130.

Tribolet R. and Sigel H. 1987b. Self-association and protonation of adenosine 5′monophosphate in comparison with its 2′- and 3′- analogues and tubercidin 5-monophosphate (7-deaza-AMP). Eur. J. Biochem. 163, 353-363.

Verrechio A., Germann M. W., Schick B. P., Kung B., Twardowski T. and San Antonio J.D. 2000. J. Biol. Chem. 17: 7701-7707.

Wakefield T. W. et al. 1992. Heparin-mediated reductions of the toxic effects of protamine sulphate on rabbit myocardium. J. Vasc. Surg. 16: 47-53.

Walsh et al. 1992. Modulation of tissue factor on human monocytes by cisplatin and adriamycin. Br. J. Haematol. 81: 480-488.

Werb Z., Vu T. H. and Rinkenberger J. L. et al. 1999. Matrix-degrading proteases and angiogenesis during development and tumor formation. APMIS 107, 11-18,

Wheeler, H. R. and Geczy C. L. 1990. Induction of macrophage procoagulant expression by cisplatin, daunomycin and doxorubucin. Int. J. Cancer 46: 626-632.

Zetter B. R. 1998. Angiogenesis and tumor metastasis. Ann. Rev. Med. 49, 407-424.

1. A compound of the general formula A-B, which in the vicinity of tumor cells or endothelial cells involved in tumor angiogenesis results in a positively charged moiety B and an uncharged or negatively charged moiety A, whereby said moiety B is able to induce blood clotting by Interacting with negatively charged heparin-like substances lining vascular endothelia and whereby the positive charge is reversibly masked by the uncharged or negatively charged moiety A in order to prevent unspecific disseminated blood coagulation and toxicity.

2. A compound according to claim 1 wherein A and B carry at least one negative charge and at least one positive charge, respectively, whereby the positive charge of B is masked by the negative charge of A.

3. A compound according to claim 1 wherein A and B are uncharged moieties in compound A-B, whereby B is able to obtain (a) positive charge(s) when released from A-B.

4. A compound according to any of the claims 1 to 3, wherein A is a masking moiety carrying a negative charge and is unstable at pH 6.0-6.5.

5. A compound according to claim 4, wherein A is a pH unstable capping moiety chosen from the group comprising citraconyl and dimethylmaleyl or a combination thereof.

6. A compound according to any of the claims 1 to 3, wherein A is of the general formula X—Y, wherein X is a neutral or preferably a negatively charged N-capping moiety and Y is a linker stable in normal tissues and body fluids degradable by enzymes which are released by tumor cells or endothelial cells involved in tumor neoangiogenesis.

7. A compound according to claim 6, wherein said N-capping moiety is chosen from the group comprising succinyl, glutaryl, maleyl and diglycolyl; a polyalkyleneglycol or a combination thereof.

8. A compound according to claim 7, wherein said polyalkyleneglycol N-capping moiety is a polyethylene glycol having an average molecular weight ranging from 100 up to 12000 Da.

9. A compound according to claim 8, wherein said polyethylene glycol has an average molecular weight of 350 Da.

10. A compound according to any of the claims 6 to 9, wherein Y is a substrate for extracellular hydrolases releasable by tumor cells or neoangiogenic endothelial cells and that is resistant to hydrolases found in normal tissues and body fluids.

11. A compound according to claim 10, wherein said hydrolase is an extracellular tumor peptidase.

12. A compound according to any of the claims 6 to 11, wherein Y is Y′-L-Leu-L-Ala-Y″ and Y′ and Y″ are amino acids or oligopeptides consisting essentially of L-amino acids.

13. A compound according to claim 12, wherein Y is N-β-Ala-L-Leu-L-Ala-L-Leu.

14. A compound according to any of the claims 1 to 13, wherein B is a covalent assembly of positively charged chemical groups.

15. A compound according to any of the claims 1 to 13, wherein B is a positively charged molecule which in aqueous solutions is able to form non-covalent polycationic aggregates.

16. A compound according to any of the claims 1 to 15, wherein the coagulant moiety B comprises a positively charged polymer.

17. A compound according to claim 16, wherein said B moiety comprises an heparin-binding peptide.

18. A compound according to claim 17, wherein the B moiety comprises a D-amino acid heparin-binding peptide.

19. A compound according to claims 17 or 18, wherein B comprises a poly-D-Lys.

20. A compound according to claims 17 or 18, wherein said heparin-binding peptide is chosen from the group comprising (AKKARA)ₙ or (ARKKAAKA)ₙ; whereby n is an integer from 1 to 10.

21. A compound according to any of the claims 1 to 15, wherein amino-groups of said coagulant moiety B are branched with at least one D-amino acid.

22. A compound according to claim 21, whereby the moiety B comprises a peptide consisting essentially of D-amino acids or branched with at least one D-amino acid.

23. A compound according to any of the claims 1 to 22, wherein the procoagulant moiety B is of the general formula U—V, wherein U is an oligopeptide consisting essentially of D-amino acids carrying (a) positive charge(s) and V is an aggregation inducer which is covalently bound to U via its terminal amino group without interference with the aggregation properties of V.

24. A compound according to any of the claims 1 to 22, wherein the procoagulant moiety B is of the general formula U—V, wherein U is an oligopeptide consisting essentially of D-amino acids carrying hydrophobic residues and V is (a) positively charged moiety(ies) which is covalently bound to U via its terminal amino group.

25. A compound according to any of the claims 1 to 22, wherein the procoagulant moiety B is of the general formula U—V, wherein U is an oligopeptide consisting essentially of D-amino acids covalently bound to V through its terminal carboxyl group, whereby both U and V carry positive charges and allowing intermolecular interactions.

26. A compound according to any of the claims 21 to 25, wherein the D-amino acids are chosen from the group comprising D-Ala, D-Val, D-Leu, D-Ile, D-Phe, D-Trp, D-Cys, D-Asn, D-Gln, D-Ser, D-Thr, D-Tyr, D-Lys and D-Arg.

27. A compound according to any of the claims 23 to 26, wherein U is a tetrapeptide of D-amino acids.

28. A compound according to claim 27, wherein U is chosen from the group comprising D-Ala-D-Leu-D-Ala-D-Leu or D-Ala-D-Lys-D-Ala-D-Leu.

29. A compound according to claim 23, wherein the aggregation inducer is chosen from the group comprising anthracyclines, acridine dyes, purine and pyrimidine derivatives, dihydroanthraquinones and nicotinamide.

30. An active compound according to any of the claim 1 to 29 wherein at the N-terminal end of the B moiety one or more additional amino acid residues are present, said residues are the result of an incomplete processing of the A moiety.

31. A compound according to any of the claims 1 to 30 for use as a medicine.

32. Use of a compound according to any of the claims 1 to 30 for the manufacture of a medicament for the treatment and/or prevention of tumor related disorders.

33. A pharmaceutical composition comprising the compound according to any of the claims 1 to 30 and a pharmaceutically acceptable adjuvant or excipient.

34. Products containing a compound according to any of the claims 1 to 30 and any other antitumoral agent as a combined preparation for simultaneous, separate or sequential use in cancer therapy.

35. Method for the treatment of an subject in need of an anti-tumor treatment administering a therapeutically effective amount of a compound, a composition or product according to any of the claims 31, 33 and 34.

36. A method for synthesizing a compound according to any of the claims 1 to 5 comprising the step of reacting a precursor of A with a precursor of B under conditions in which a reactive group of A condenses with a complementary reactive group of B, thereby forming A-B.

37. A method for synthesizing a compound according to any of the claims 6 to 22 comprising the steps of:
1) reacting a precursor of X with a precursor of Y* under conditions In which a reactive group of X condenses with a complementary reactive group of Y*, thereby forming X—Y*, whereby Y* is a precursor of Y comprising a reactive group*, and,
2) reacting X—Y* with a precursor of B under conditions in which a reactive group of X—Y* condenses with a complementary reactive group of the precursor of B thereby forming X—Y—B.

38. A method for synthesizing a compound according to any of the claims 23 to 29 comprising the steps of:
1) reacting a precursor of *U with a precursor of V under conditions in which a reactive group of *U condenses with a complementary reactive group of V, thereby forming *U—V, whereby *U Is a precursor of U comprising a reactive group*, and,
2) reacting *U—V with a precursor of A under conditions in which a reactive group of *U—V condenses with a complementary reactive group of the precursor of A thereby forming A-U—V.

39. A method for synthesizing a compound according to any of the claims 6 to 29 comprising the steps of:
1) reacting a precursor of X with a precursor of Y* under conditions in which a reactive group of X condenses with a complementary reactive group of Y*, thereby forming X—*, whereby Y* is a precursor of Y comprising a reactive group *, and,
2) reacting a precursor of *U with a precursor of V under conditions in which a reactive group of *U condenses with a complementary reactive group of V, thereby forming *U—V, whereby *U is a precursor of U comprising a reactive group *, and,
3) reacting X—Y* with *U—V under conditions in which a reactive group of X—Y* condenses with a complementary reactive group of *U—V thereby forming X—Y—U—V.
